SEQUENCE LISTING (41 sequences)

SEQ ID NO: 1 - protein, 1374 aa, Schizosaccharomyces pombe
MDISSFLLPQLLRKYQQDVYNIASKQNTLLVMRTGAGKTLLAVKLIKQKLEEQILIQESNLEHKKISVFLVNKVPLVFQQAEYIRSQLPAKVGMFYGELSIEMSEQLLTNIILKYNVIVITADLFYLFLARGFLSINDLNLIIFDECHHAIGNDAYARIMNDFYHRAKAVLSKKHFTLPRIFGMTASPFTGKKGNLYHRLYQWEQLFDSKAHVVSENELADYFCLPEESYVMYSNKLVVPPSDSIIKKCEETLQGCKLISRAVKTALAETIDMGLWFGEQVWLYLVDFVETKRLKKKALGKQLSDDEELAIDRLKIFVEDWKNNKYSDNGPRIPVFDSTDVTDKVFKLLELLKATYRKSDSVRTVIFVERKATAFTLSLFMKTLNLPNIRAHSFIGHGPSDQGEFSMTFRRQKDTLHKFKTGKYNVLIATAVAEEGIDVPSCNLVIRFNICRTVTQYVQSRGRARAMASKFLIFLNTEELLIHERILHEEKNLKFALSELSNSNIFDSLVCEERERVTDDIVYEVGETGALLTGLYAVSLLYNFCNTLSRDVYTRYYPTFTAQPCLSGWYCFEVELPKACKVPAAQGSPAKSIRKAKQNAAFIMCLDLIRMGLIDKHLKPLDFRRKIADLETLEEDELKDEGYIETYERYVPKSWMKVPEDITRCFVSLLYTDANEGDNHIFHPLVFVQAHSFPKIDSFILNSTVGPRVKIVLETIEDSFKIDSHLLELLKKSTRYLLQFGLSTSLEQQIPTPYWLAPLNLSCTDYRFLENLIDVDTIQNFFKLPEPVQNVTDLQSDTVLLVNPQSIYEQYAFEGFVNSEFMIPAKKKDKAPSALCKKLPLRLNYSLWGNRAKSIPKSQQVRSFYINDLYILPVSRHLKNSALLIPSILYHIENLLVASSFIEHFRLDCKIDTACQALTSAESQLNFDYDRLEFYGDCFLKLGASITVFLKFPDTQEYQLHFNRKKIISNCNLYKVAIDCELPKYALSTPLEIRHWCPYGFQKSTSDKCRYAVLQKLSVKRIADMVEASIGACLLDSGLDSALKICKSLSVGLLDISNWDEWNNYFDLNTYADSLRNVQFPYSSYIEETIGYSFKNKKLLHLAFIHPSMMSQQGIYENYQQLEFLGDAVLDYIIVQYLYKKYPNATSGELTDYKSFYVCNKSLSYIGFVLNLHKYIQHESAAMCDAIFEYQELIEAFRETASENPWFWFEIDSPKFISDTLEAMICAIFLDSGFSLQSLQFVLPLFLNSLGDATHTKAKGDIEHKVYQLLKDQGCEDFGTKCVIEEVKSSHKTLLNTELHLTKYYGFSFFRHGNIVAYGKSRKVANAKYIMKQRLLKLLEDKSNLLLYSCNCKFSKKKPSDEQIKGDGKVKSLT

SEQ ID NO: 2 - protein, 137 aa, Schizosaccharomyces pombe
MGSSHHHHHHSSGLVPRGSHMKGDIEHKVYQLLKDQGCEDFGTKCVIEEVKSSHKTLLNTELHLTKYYGFSFFRHGNIVAYGKSRKVANAKYIMKQRLLKLLEDKSNLLLYSCNCKFSKKKPSDEQIKGDGKVKSLT

SEQ ID NO: 3 - protein, 104 aa, Laccaria bicolor (strain S238N-H82)
LQTLSHHPTKILFELFQAQGCHLFEISKENNAEKTLCHVLVHEVILASAEDVMPTLAARQASIFALDALQGDAEFMRRICDCRTQSSNKKPRRKSEFEEELLTL

SEQ ID NO: 4 - protein, 106 aa, Coprinopsis cinerea (strain Okayama-7 / 130 / FGSC 9003)
LRTLTHHPTKTLFEYIQAHGCQQLKLSKGPQQKGGKMSCEILVHETVCASASDVSLSVAAREASTRALDRFKEDGDFLFKLCDCRTGGQNKGGQVNKGFEHALSSL

SEQ ID NO: 5 - protein, 103 aa, Schizophyllum commune
MNTLAVHPTKILLELVQSQCCHKLELVKSASAGQTQCNVLLHGAVIASKTAVNGVLAARGASSIALNKLDRDPGLVARTCTCRTAGMGKAERDVDQFLSEFKA

SEQ ID NO: 6 - protein, 121 aa, Podospora anserina
YDTFANKHPVTFLGHEMQHRFRCQEWRLLTREIMVDKDEEGGMMGEKQVVCAVMVHGQKLAHAVAQSARYSKIGAAKKALRELEGLDQGEFRRRVGCDCKVVGEVEGKDGEKGEEVEIEVY

SEQ ID NO: 7 - protein, 122 aa, Chaetomium globosum
YDAFANKHPVTFLAGIMQGRMRCAEWRLLVKDLPPVVGAGAGTELETPQVVCAVRVHGLTLAHAVAASGRYGKIAAAKKAIQVLEGMDAEAFRKAYGCACVLGEEEQQAAQTIATELEVFPA

SEQ ID NO: 8 - protein, 109 aa, Verticillium albo-atrum (strain VaMs.102)
YDSFASSQPVTTLARMMQQDFGCQDWRLLVSELPPSCEDGGATAITETEVICGFMVHGRILLHAKSSSGQYAKVGAAKRAVEKLMGLGNDKEAFREDFGCDCGCDGQAI

SEQ ID NO: 9 - protein, 134 aa, Neurospora crassa
YDTFANKHPVTFVANMMAHKFRCNEWRSFAKELDTDVTEGRGGRGGNGAVAGEISEINPPKVVSALLVHGKTVVHAVAASGRYAKSAMAKKAIKLLEGMSVEEFRERLGCNCKGVPMEVDGGVPEADVDGEVHG

SEQ ID NO: 10 - protein, 132 aa, Sordaria macrospora
YDSFANKHPVTFVANMMAHKFCCQEWRSLAKELDTDVSEGRGGRGGAVAGEITEINPPKVVSALLVHGKTVAHAVAASGRYAKSAMAKKAIKMLEGMTVEEFREKLGCDCKGVPMEVDGGVAAEADVVDEDG

SEQ ID NO: 11 - protein, 117 aa, Nectria haematococca
YDSFANKHPVTYLSKKMQQDLGCTNWRVSAENVPCGVEEGIAALTESDVVAVFMVHQKVVANATAKSGRYAKIAVAKRALAMLDEHEGDFETMKRALGCDCKPGAVQDDVNDHGTAI

SEQ ID NO: 12 - protein, 114 aa, Cryphonectria parasitica
YDTFANKHPVTFFTQYVFETFGCHAYGLHAEEMPVKDDTGLVTGKTQVVAGILLHGQVVEGAVRDSGRYAKIAAARKALDKLRSMTRQEFLDAYKCDCKPGEAAEDISESATAI

SEQ ID NO: 13 - protein, 121 aa, Magnaporthe grisea
YDMYSSKHPVTHITSIITTQFGCSSFRLMVHEIPDDVQGEGLVTGAVKVVAACMIHGEVRCHAVAASGRYAKLAVAKQAVAIYEDMSPTEFRLRHGCNCKPEDGDGDHGVLVDHRADCEPE

SEQ ID NO: 14 - protein, 103 aa, Coccidioides posadasii (strain C735)
YDTYANKHPTTYLHNRLAIDFGCSNYCLKAGEVPYVDGPGTRVLAAVIVHDEVVTEGVASSSRYAKLKASEAALSKLEGLPPFRFREIYGCNCQAGQSEAGNE

SEQ ID NO: 15 - protein, 103 aa, Coccidioides immitis
YDTYANKHPTTYLHNRLAIDFGCSNYCLKAGEVPYVDGPGTRVLAAVIVHDEVVAEGVASSSRYAKLKASEAALSKLEGLPPFRFREIYGCNCQAGQSEAGNE

SEQ ID NO: 16 - protein, 113 aa, Ajellomyces dermatitidis
YDTFANKHPTTYFHNRLTIDFGCTSYCLKAGEVQMVDGTEPRVYAAVIVHDEFVAEGIASSVRYAKIKASEAALAKLEGLAPFKFREIYGCDCVVNKGEEENCSLKNVKPICG

SEQ ID NO: 17 - protein, 115 aa, Ajellomyces capsulata (strain H143)
YDAFANKHPTTYFHNRLTIDFGCANYCLKAGEVQMVDSTEPRIYAAVIVHDKFVAQGIASSVRYAKTKASEAALAKLEGLASFKFREIYGCNCAADKVEEATDAAKRIVDEAKAA

SEQ ID NO: 18 - protein, 110 aa, Arthroderma benhamiae (strain CBS 112371)
YDTFANKHPITFLHTRLAVDFGCTFYALKADEIPCVDGTTVRALAAVCVHDDIVAEGVASSPRHAKLKASQNALQILEEMSAADFRAKYKCDCRERQEEHEEEEPETAEE

SEQ ID NO: 19 - protein, 110 aa, Trichophyton verrucosum (strain HKI 0517); identical to SEQ ID NO: 18

SEQ ID NO: 20 - protein, 110 aa, Nannizzia otae (strain CBS 113480)
YDTFANKHPITFLHTRLAVDFGCTFYALKADEIPCVDGTTIRALAAVCVHNDIVAEGVATSPRHAKLKASQNALQILEQMSTAEFRATYKCDCREKQEEREEGELEKQNN

SEQ ID NO: 21 - protein, 108 aa, Aspergillus flavus
  misc_feature: strain ATCC 200026 / FGSC A1120 / NRRL 3357 / JCM 12722 / SRRC 167
YDTFANKHPTTFLQNRLTNEYGCTNYCLKAGEIPVVDGGTVSVLAAVIVHEVVIAEGTASSGRYAKVKASEKALSVLENMGPSEFREKYHCDCRTANDSQPMDIGTAI

SEQ ID NO: 22 - protein, 108 aa, Aspergillus oryzae
YDTFANKHPTTFLQNRLTNEYGCTNYCLKAGEIPVVDGGTVSVLAAVIVHEVVIAEGTASSGRYAKVKASEKALSVLENMGPSEFREKYHCDCRTANGSQPMDIGTAI

SEQ ID NO: 23 - protein, 108 aa, Aspergillus niger (strain CBS 513.88 / FGSC A1513)
YDTFANKHPTTFLHNRLTNEFGCVNYCLKAGEMPSIDGAPAGVLAAVIVHDVVIAEGTATSGRYAKVKASEKALAVLDEISSAEFQRKFRCDCRESGDSARLDIGTAI

SEQ ID NO: 24 - protein, 109 aa, Neosartorya fischeri
  misc_feature: strain ATCC 1020 / DSM 3700 / FGSC A1164 / NRRL 181
YDTFANKHPTTFLHNKLTNEYGCTNYCLKAGELPTIDGAPAGVLAAVIVHGNVISEARSSSSRYAKVKASEKALAVLDGLLPFEFCQKYHCGCKETQNSSSAVEIGTAI

SEQ ID NO: 25 - protein, 109 aa, Aspergillus fumigatus
YDTFANKHPTTFLHNKLTNEYGCTNYCLKAGELPTIDGAPATVLAAVIVHGNVISEARSSSSRYAKITASEKALAVLDGLLPSEFCQKYRCDCKETKNSSSVVEIGTAI

SEQ ID NO: 26 - protein, 109 aa, Aspergillus clavatus
YDTFANKHPTTFLHNRLANEFGCTNYCLKAGEMPAIDGMPAGVLAAVIVHNSVVSEATASSSRYAKIRASERALVVLDGLLPYEFRQRYNCNCQVVGNPASAPDIGTAI

SEQ ID NO: 27 - protein, 109 aa, Aspergillus terreus (strain NIH 2624 / FGSC A1156)
YDTFANRHPTTFLHNKLTNEYGCLNYCLKAGEIPGADGDASTVLAAVIVHDTILTTGVASSGRYAKVKASENALTELLHIDRNEFRKRYQCDCVQENGEHGERDVGTPI

SEQ ID NO: 28 - protein, 113 aa, Penicillium marneffei (strain ATCC 18224 / CBS 334.59 / QM 7333)
YDTFANKHPTTFLHGKLTDDFGCIQYTLKAAEIPSVDGAPTVVLAAVLVHDVDLAQATASSGRYAKIKASEVALKNLEGLSAEAFRQKYHCQCSRTLSNGAVPPLQEDIGCAI

SEQ ID NO: 29 - protein, 115 aa, Talaromyces stipitatus
  misc_feature: strain ATCC 10500 / CBS 375.48 / QM 6759 / NRRL 1006
YDTFANKHPTTFLHRKLTDDFGCIQYTLRAAEIPSVDGAPPVVLAAVLVHDVVLAQGTASSGRYAKIKASEVAMKNLEGLSVSRFREKYHCKCSRNVSSHGAAAPPRPEDMGCAI

SEQ ID NO: 30 - protein, 109 aa, Penicillium chrysogenum
  misc_feature: strain ATCC 28089 / DSM 1075 / Wisconsin 54-1255
YDTFANRHPTTFLHSQMTHNYRCADYCLKSAEVPAVEGETPRVFAAVMVHGQSIASAVASSSRYAKVRASTRALAVIDGMSLSEFRERYHCACQGGQVAVDHANIGTAV

SEQ ID NO: 31 - protein, 115 aa, Botryotinia fuckeliana (strain B05.10)
YDTYANKHPTTFLTNFLQKNMGCEDWAPVSKEVPGEDGRKNVVVCGVIIHNKVVSTATAESMRYARVGAARNALRKLEGMSVREFRDEYGCSCEGDVVDEEGNIEFVEREDGMEG

SEQ ID NO: 32 - protein, 115 aa, Sclerotinia sclerotiorum (strain ATCC 18683 / 1980 / Ss-1)
YDTYANKHPTTFLTNFLQKNMGCEDWAPVSREMPGEDGRKNVVVCGVIVHNKVVSTFQAESMRYAKVGAAKKALAQLEGMSVREFREQFECSCKGDVADAEGNVEFVEREDGISG

SEQ ID NO: 33 - protein, 115 aa, Pyrenophora tritici-repentis (strain Pt-1C-BFP)
YENFASNHPTTRLSRILSINFGCNDWRMGALEMETLIPGKGKAIAAMIMIHGKVHFYSLGQSGRYARVRVSHAALEKLDGLPPFEFRKKYGCYCAEHGDEMAKGDQMREAMGTSI

SEQ ID NO: 34 - protein, 116 aa, Phaeosphaeria nodorum
YENFASNHPTTRLSRLLSINFGCSEWRMGALETETLIPGKGKAIAAMVMIHDKVHFHSLGQSGRYARVRASHAALEKLEGLPPYEFRSKYGCDCVDEGEGEAGVDEAAVSKKVELM

SEQ ID NO: 35 - protein, 107 aa, Tuber melanosporum
FDNFAGQHPTTYITKFMDDLNCEFWGFESQQCMTGNMTYILTAIVVHREIFSYGSGVSVKAARVEASLAAIQKLEDPEGLRLLKEICNCAEVREKRAKEKQDKGKIN

SEQ ID NO: 36 - protein, 98 aa, Cryptococcus neoformans
GPSASDLHPKGLLTRWMQAKGCAYWKLDAVGPKLCEGVVTCHDQDVARCKAFTTHVAVRNVCEEAIKRLRDENRIKDICDCPSFKEIVPEDKTFGKWR

SEQ ID NO: 37 - protein, 109 aa, Mucor circinelloides f. lusitanicus
TPELIKFHPLRKLITDLQRFGCDSFMLRNHGSGETGPESQKCVIFLHDKPLATGSDWNIKTARRHAATKASQRLADEPGLLESICNCRVSMIKRGLMKDDRLQKNLDDD

SEQ ID NO: 38 - protein, 104 aa, Schizosaccharomyces japonicus (strain yFS275 / FY16936)
YEGVFNEPPVFSQINQHLQKQGCKQYKFQCVIIDEEEKSDLLYTKAVTHHYEYSLIIHDEIVCSAKALNKKNSQKLLSLALKELYQKASVRLFRKCECQLSNSD

SEQ ID NO: 39 - protein, 121 aa, Schizosaccharomyces pombe
THTKAKGDIEHKVYQLLKDQGCEDFGTKCVIEEVKSSHKTLLNTELHLTKYYGFSFFRHGNIVAYGKSRKVANAKYIMKQRLLKLLEDKSNLLLYSCNCKFSKKKPSDEQIKGDGKVKSLT

SEQ ID NO: 40 - protein, 109 aa, Coprinopsis cinerea (strain Okayama-7 / 130 / FGSC 9003)
LSLDMAKDPVSLLLEWVAQHGCREISFSKNRKFNYDAGVVLEVHGKQVVGPIVASSPGVAKFIASERALSILTDESNELALSKLCDCEENRQNMEIDVDAALAEMKVVT

SEQ ID NO: 41 - protein, 112 aa, Postia placenta (strain ATCC 44394 / Madison 698-R)
LSPNLPRDPISELMIWVSRSGCSKASFKKSHSNAEAKKNDSISVIVHDKTVVGPLFAPNLSLAKGLASERARSILEDPKSPFYLKRICSCGTSHETEGLDDETEEGFARLAR

(19) United States
(12) Patent Application Publication
(10) Pub. No.: US 2014/0093506 A1
(43) Pub. Date: Apr. 3, 2014

(54) ANTI-FUNGAL-AGENTS

(71) Applicants: Marc Buehler, Riehen (CH); Stephan Emmerth, Basel (CH); Frederic Hai-Trieu Allain, Zurich (CH); Pierre Barraud, Zurich (CH)

(72) Inventors: Marc Buehler, Riehen (CH); Stephan Emmerth, Basel (CH); Frederic Hai-Trieu Allain, Zurich (CH); Pierre Barraud, Zurich (CH)

(21) Appl. No.: 13/885,291

(22) PCT Filed: Nov. 14, 2011
(86) PCT No.: PCT/EP11/70022

§ 371 Date: May 16, 2013

(30) Foreign Application Priority Data
Nov. 15, 2010 (EP) 10191173.3

(51) Int. Cl. C07K 16/40; G01N 33/573

(52) U.S. Cl. CPC C07K 16/40; G01N 33/573
USPC 424/139.1; 530/387.9; 435/7.4

(57) ABSTRACT

The present application relates to methods for reducing growth of a fungus having a coordination complex formed at the C-terminus of its dicer (dicer 1, ribonuclease type III) between an anion and the amino acids corresponding to the amino acids C1275, H1312, C1350 and C1352 of Dcr1 of S. pombe (fission yeast) comprising the step of contacting a fungal cell with an effective amount of an agent, wherein said agent disrupts the coordination complex formed at the C-terminus of fungal dicer between an anion and the amino acids corresponding to the amino acids C1275, H1312, C1350 and C1352 of Dcr1 of S. pombe.

### FIELD OF THE INVENTION

The present invention relates to methods of reducing the growth of a fungus.

### BACKGROUND OF THE INVENTION

Invasive fungal infections are well recognized as diseases of the immunocompromised host. Over the last twenty years there have been significant rises in the number of recorded instances of fungal infection (Groll et al., 1996, J Infect 33, 23-32). This rise is partly due to increased awareness and improved diagnosis of fungal infection. However, the primary cause of this increased incidence is the vast rise in the number of susceptible individuals. This is due to a number of factors including new and aggressive immunosuppressive therapies, increased survival in intensive care, increased numbers of transplant procedures, and the greater use of antibiotics worldwide. In certain patient groups, fungal infection occurs at high frequency. For instance, lung transplant recipients have a frequency of up to 20% colonization and infection with a fungal organism and fungal infection in allogenic haematopoetic stem transplant recipients is as high as 15% (Ribaud et al., 1999, Clin Infect Dis. 28:322-30). Currently only four classes of antifungal drug are available to treat systemic fungal infections. These are the polyenes (e.g., amphotericin B), the azoles (e.g., ketoconazole or itraconazole) the echinocandins (e.g., caspofungin) and flucytosine. The polyenes are the oldest class of antifungal agent being first introduced in the 1950's. The exact mode of action remains unclear but polyenes are only effective against organisms that contain sterols in their outer membranes. It has therefore been proposed that amphotericin B interacts with membrane sterols to produce pores allowing leakage of cytoplasmic components and subsequent cell death. Azoles function by the inhibition of 14α-demethylase via a cytochrome P450-dependent mechanism. This leads to a depletion of the membrane sterol ergosterol and the accumulation of sterol precursors resulting in a plasma membrane with altered fluidity and structure.

Echinocandins work by inhibiting the cell wall synthesis enzyme β-glucan synthase, leading to abnormal cell wall formation, osmotic sensitivity and cell lysis.

Flucytosine is a pyrimidine analogue interfering with cellular pyrimidine metabolism as well DNA, RNA and protein synthesis. However widespread resistance to flucyotosine limits its therapeutic use.

It can be seen that, to date, the currently available antifungal agents act primarily against only two cellular targets; membrane sterols (ployenes and azoles) and β-glucan synthase (echinocandins).

Resistance to both azoles and polyenes has been widely reported leaving only the recently introduced echinocandins to combat invasive fungal infections. As the use of echinocandins increases, resistance by fungi will inevitably occur.

The identification of new classes of anti-fungal agent with novel modes of action is hence required to ensure positive therapeutic outcomes for patients in the future. Novel fungal-specific mechanisms are likely to present the best opportunity for the development of effective novel anti-fungal agents. In particular it is highly desirable that target of a new therapy is present in a range of fungi, but absent from humans.

In addition, anti-fungal agents are also of interest for many other uses, for instance in agriculture where fungi can cause a lot of damages or in coating to prevent e.g. mildew.

RNAseIII ribonucleases act at the heart of RNA silencing pathways by processing precursor RNAs into mature microRNAs and siRNAs. In the fission yeast Schizosaccharomyces pombe, siRNAs are generated by the RNAseIII enzyme Dcr1 and are required for heterochromatin formation at centromeres. In an earlier study (Emmerth et al., 2010, Dev. Cell 18, 102-113), some of the present inventors analyzed the subcellular localization of Dcr1 and noted that it accumulates in the nucleus and is enriched at the nuclear periphery. They moreover noted that nuclear accumulation of Dcr1 depends on a short motif which impedes nuclear export promoted by the double-stranded RNA binding domain of Dcr1. Absence of this motif renders Dcr1 mainly cytoplasmic and is accompanied by remarkable changes in gene expression and failure to assemble heterochromatin.

### SUMMARY OF THE INVENTION

In further studies, the present inventors elucidated the structure of the relevant short motif of Dcr1. The present inventors noted that the action of said short motif of Dcr1 is surprisingly due to a particular structural conformation induced by a coordination complex between a cation, e.g. zinc, and 4 amino acids. The present inventors further found out that this coordination complex is present in all the fungus they investigated and that disruption of said coordination complex leads to the death of the fungal cells.

The present invention thus encompasses a method for reducing growth of a fungus having a coordination complex formed at the C-terminus of its dicer (dicer 1, ribonuclease type III) between a cation and the amino acids corresponding to the amino acids C1275, H1312, C1350 and C1352 of SEQ ID NO:1 comprising the step of contacting a fungal cell with an effective amount of an agent, wherein said agent disrupts the coordination complex formed at the C-terminus of fungal dicer between a cation and the amino acids corresponding to the amino acids C1275, H1312, C1350 and C1352 of SEQ ID NO:1. In some embodiments of the invention, the cation is selected from the group consisting of zinc, magnesium, manganese, cobalt, copper and iron. In some embodiments of the invention, the agent is an antibody or a small molecule. In some embodiments, the method of the invention is a method for treating a subject having, or being at risk of having, a fungal infection comprising the further step of administering the antifungal agent to the subject. Said subject can be a mammal, for instance a human subject or a non-human subject, or a plant or a seed.

In some embodiments of the invention, fungal cell is from the ascomycota phylum and is, for instance, selected from the group comprising Coccidioides immitis, Aspergillus niger, Aspergillus clavatus, Aspergillus oryzae, Aspergillus nidulans, Aspergillus terreus, Neosartorya fischeri, Aspergillus fumigatus, Phaeosphaeria nodorum, Magnaporthe grisea, Neurospora crassa, Chaetomium globosum, Cryphonectria parasitica, Schizosaccharomyces pombe, Cryptococcus neoformans, Coccidioides posadasii, Ajellomyces dermatitidis, Ajellomyces capsulate, Arthroderma benhamiae, Aspergillus flavus, Penicillium marneffei, Trichophyton verrucosum, Nectria haematococca, Verticillium albo-atrum, Botryotinia fuckeliana, Sclerotinia sclerotiorum, Pyrenophora tritici-repentis, Mucor circinelloides f. lusitanicus, Postia placenta, Laccaria bicolor, Coprinopsis cinerea, Schizophyllum commune H4-8, Sordaria macrospora, Podospora anserine, Nannizzia otae, Talaromyces stipitatus, Penicillium chrysogenum, Tuber melanosporum (Truffle), Schizosaccharomyces japonicas and Coprinopsis cinerea. In some embodiments, the fungal cell is from a fungus which is pathogenic for animals and is, for instance, Aspergillus fumigatus, Cryptococcus neoformans, Chaetomium globosum, Coccidioides posadasii, Coccidioides immitis, Ajellomyces dermatitidis, Ajellomyces capsulate, Arthroderma benhamiae, Aspergillus flavus, Aspergillus oryzae, Aspergillus niger, Neosartorya fischeri, Aspergillus clavatus, Aspergillus terreus, Penicillium marneffei, Trichophyton verrucosum or Nectria haematococca. In other embodiments, the fungus is a plant pathogen and is selected from a group comprising Nectria haematococca, Verticillium albo-atrum, Cryphonectria parasitica, Magnaporthe grisea, Botryotinia fuckeliana, Sclerotinia sclerotiorum, Pyrenophora tritici-repentis, Phaeosphaeria nodorum, Mucor circinelloides f. lusitanicus, and Postia placenta. A further embodiment of the invention encompasses an antibody specifically binding to an epitope at the C-terminus of a fungal dicer (dicer 1, ribonuclease type III), said epitope comprising one or more amino acid corresponding to C1275, H1312, C1350 and/or C1352 of SEQ ID NO:1, wherein the binding of said antibody to said epitope disrupts the coordination complex formed between a cation and the amino acids corresponding to C1275, H1312, C1350 and C1352 of SEQ ID NO:1 at the C-terminal of said fungal dicer.

Another embodiment of the invention encompasses an antibody, or antibody fragment, able to compete with an antibody specifically binding to an epitope at the C-terminus of a fungal dicer (dicer 1, ribonuclease type III), said epitope comprising one or more amino acid corresponding to C1275, H1312, C1350 and/or C1352 of SEQ ID NO:1, wherein the binding of said antibody to said epitope disrupts the coordination complex formed between a cation and the amino acids corresponding to C1275, H1312, C1350 and C1352 of SEQ ID NO:1 at the C-terminal of said fungal dicer.

The present invention further encompasses a method for the identification of a substance capable of disrupting the coordination complex formed between a cation and the amino acids corresponding to the amino acids C1275, H1312, C1350 and C1352 of SEQ ID NO:1 at the C-terminus of fungal dicer (dicer 1, ribonuclease type III), said method comprising the step of a) contacting said fungal dicer, or a fragment thereof wherein said fragment comprises at least the amino acids corresponding to the amino acids C1275, H1312, C1350 and C1352 of SEQ ID NO:1 and is able to form a coordination complex in presence of a cation, with a candidate substance, and b) assessing whether the coordination complex formed between the cation and the amino acids corresponding to the amino acids C1275, H1312, C1350 and C1352 of SEQ ID NO:1 is disrupted in the presence of the substance; wherein a disruption of said coordination complex indicates that said substance is a potential anti-fungal agent. In some embodiments, the integrity of said coordination complex is assessed by measuring the ability of dicer to migrate from the nucleus to the cytoplasm, for instance by use of a reporter functionally fused to said dicer. The integrity of said coordination complex can also be assessed using FRET.

The present invention also encompasses a composition comprising an antibody, or antibody fragment, according to the invention, or an agent identified using a method of the invention. Said compositions can be used as a medicament to treat a subject having, or being at risk of having, a fungal infection, or as a fungicide to treat plants having, or being at risk of having, a fungal infection.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

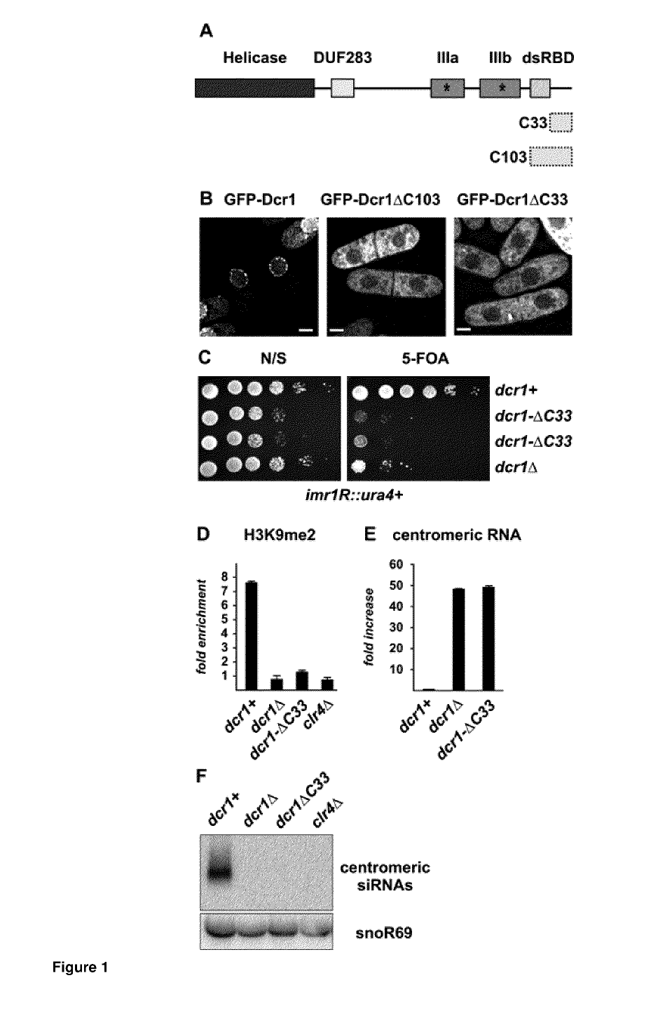

FIG. 1: The 33 most C-terminal amino acids restrict Dcr1 to the nucleus and are essential for H3K9 methylation and silencing
(A) Domain architecture of S. pombe Dcr1 (not drawn to scale). C33 and C103 indicate the last 33 and 103 amino acids of Dcr1, respectively. (B) Live-cell imaging of full length and C-terminal truncations of GFP-Dcr1. Scale bars=2 μm. (C) Silencing assay with Dcr1ΔC33 expressed from its endogenous promoter showing that the truncated protein cannot silence a heterochromatic ura4+ reporter gene (imr1R::ura4+). (D) ChIP experiment showing that the C33 truncated version of Dcr1 expressed from its endogenous promoter looses H3K9me2 of a heterochromatic ura4+ transgene (imr1R::ura4+). Fold-enrichment values from one representative experiment, normalized to act1+, are shown. The value for clr4Δ cells was set to 1. (E) Quantitative real-time RT-PCR with the same strains as in (D) showing that the C33 truncated version of Dcr1 cannot silence cendh centromeric repeats. The value for wild type cells (dcr1+) was set to 1. (D, E) Error bars represent standard deviations (STDEV). (F) Northern blot analysis performed with total RNA preparations enriched for RNAs <200 nt from the same strains as in (D). The membrane was probed with 5′ end-labeled DNA oligos specific for centromeric siRNAs, and the loading control snoR69.

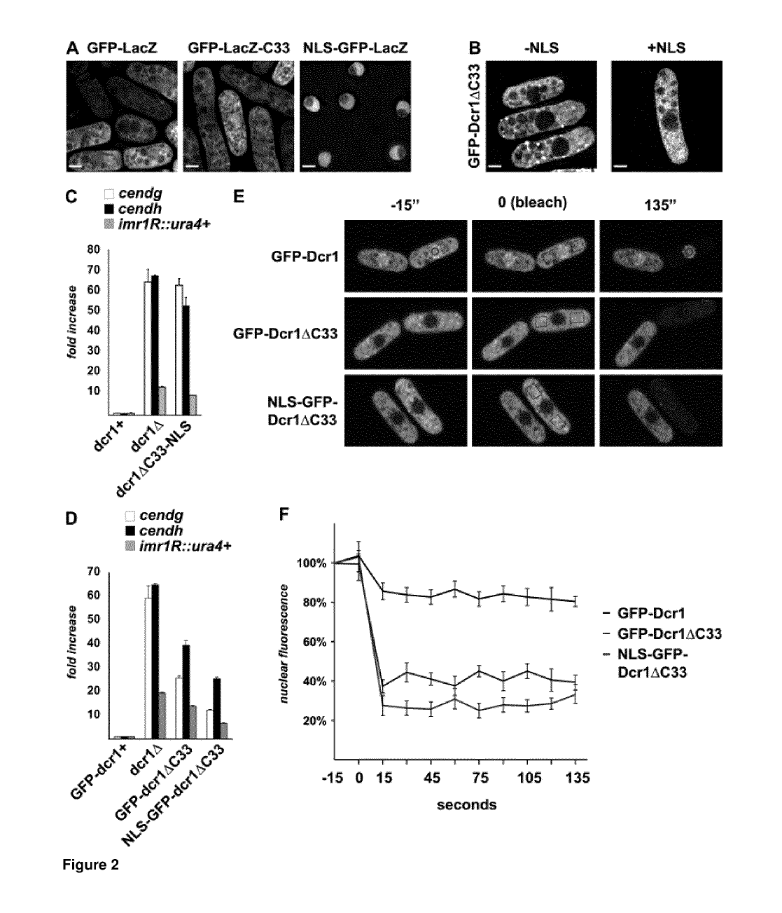

FIG. 2: Dcr1dΔC33 is a shuttling protein
(A) Live cell imaging of wild type strains transformed with the indicated plasmids. (B) Live cell imaging of GFP-Dcr1ΔC33 and NLS-GFP-Dcr1ΔC33 expressing cells. Scale bars=2 μm. (C) Quantitative real-time RT-PCR showing that Dcr1ΔC33-NLS fails to silence centromeric repeats when expressed from its endogenous promoter. (D) Quantitative real-time RT-PCR showing that moderate overexpression of GFP-Dcr1ΔC33 or NLS-GFP-Dcr1ΔC33 results in partial rescue of silencing. cendg, cendh and imr1R::ura4+ RNA levels are shown relative to dcr1+ cells and were normalized relative to act1+ RNA. (C, D) Error bars represent standard deviations (STDEV). (E, F) “Nuclear fluorescence loss in (cytoplasmic) photobleaching” (FLIP) of different GFP-tagged Dcr1 alleles. (E) Representative images of FLIP. (F) The mean fluorescence of the nucleus was determined (illustrated by circle in E, Ø˜1 μM) 15 sec before, immediately before, and three times after photobleaching the cytoplasm in 15 sec intervals (rectangle=bleach area; bleach iterations=50). Each value represents the average of 3 individual recordings. Error bars represent standard error of the mean (StDev/SQRT(n)).

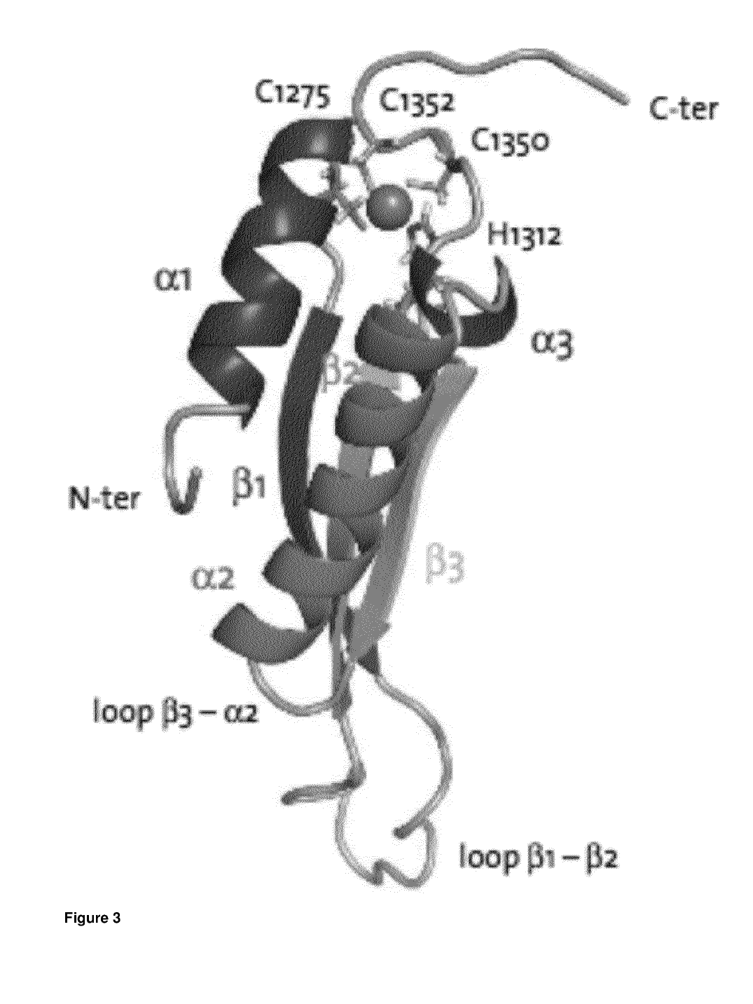

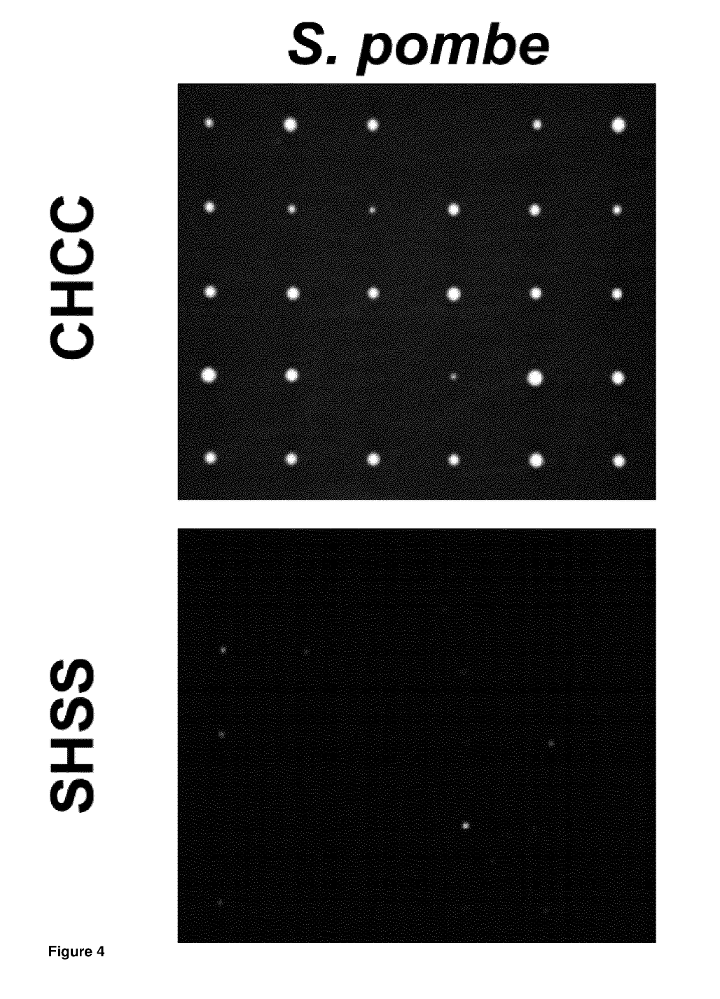

FIG. 3: Structure with coordination complex.
Cartoon representation of the Dcr1 C-terminal domain structure. The four zinc ligands (C1275, H1312, C1350 and C1352) are represented as sticks. The zinc ion is represented as a sphere.

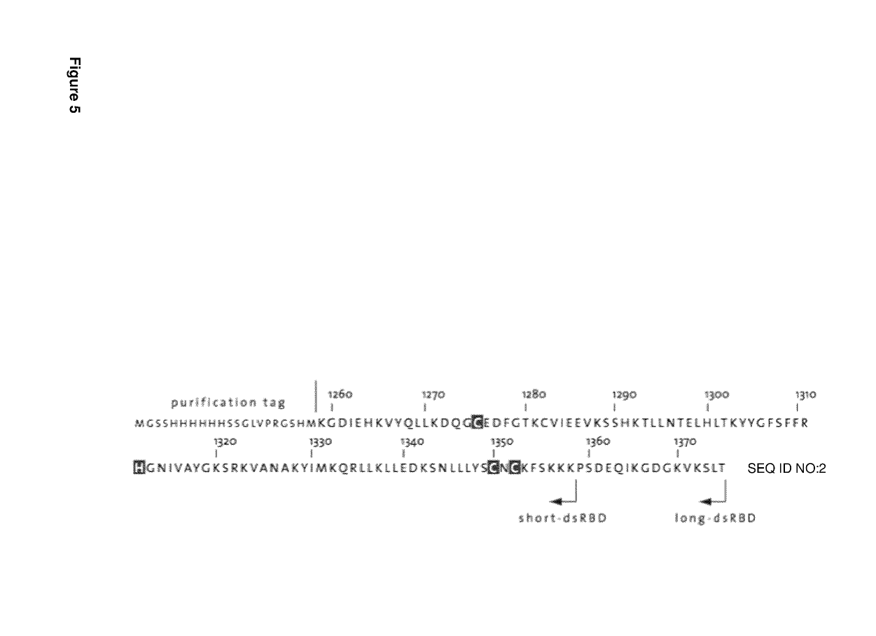

FIG. 4: Retarded growth phenotype of coordination complex mutants.
Wild type (CHCC) and coordination complex mutant (SHSS) cells were dissected onto a 5×6 matrix on YE plates and incubated at 30° C.

FIG. 5: Conserved CHCC motif in C-terminal dsRBD of the fungi DICER family.
Protein constructs of the S. pombe C-terminal domain of dcr1 used in the present application (SEQ ID NO:2). The CHCC zinc binding motif is highlighted.

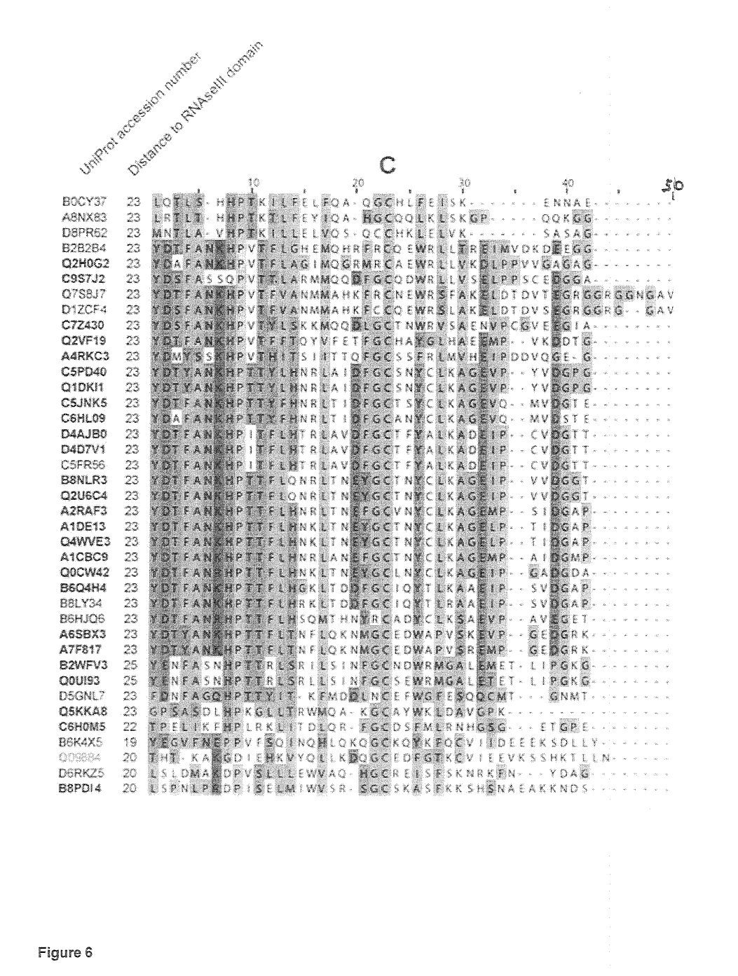

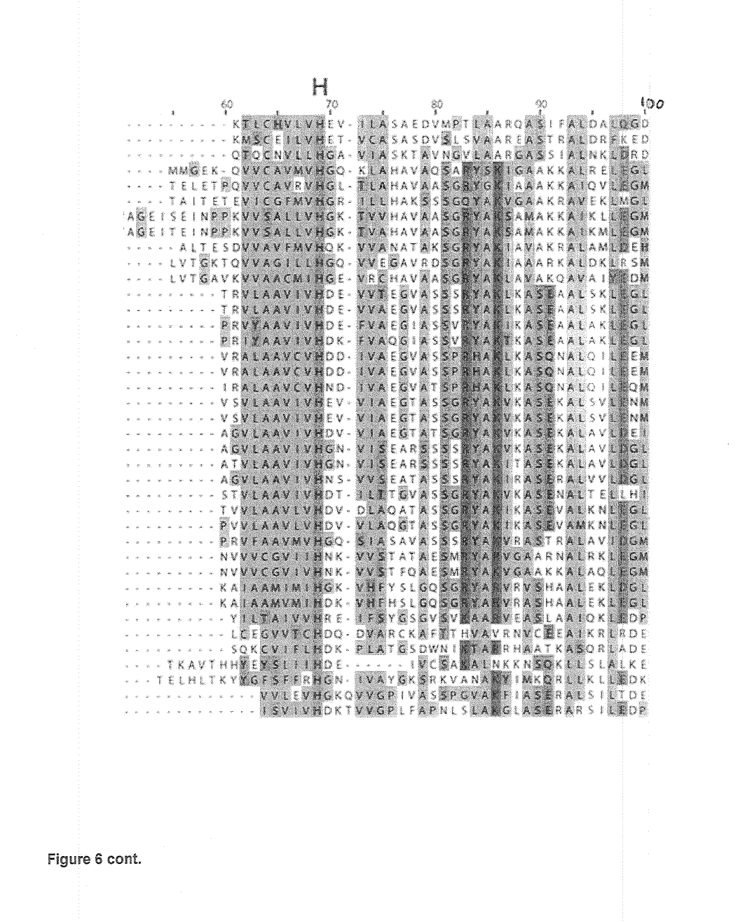

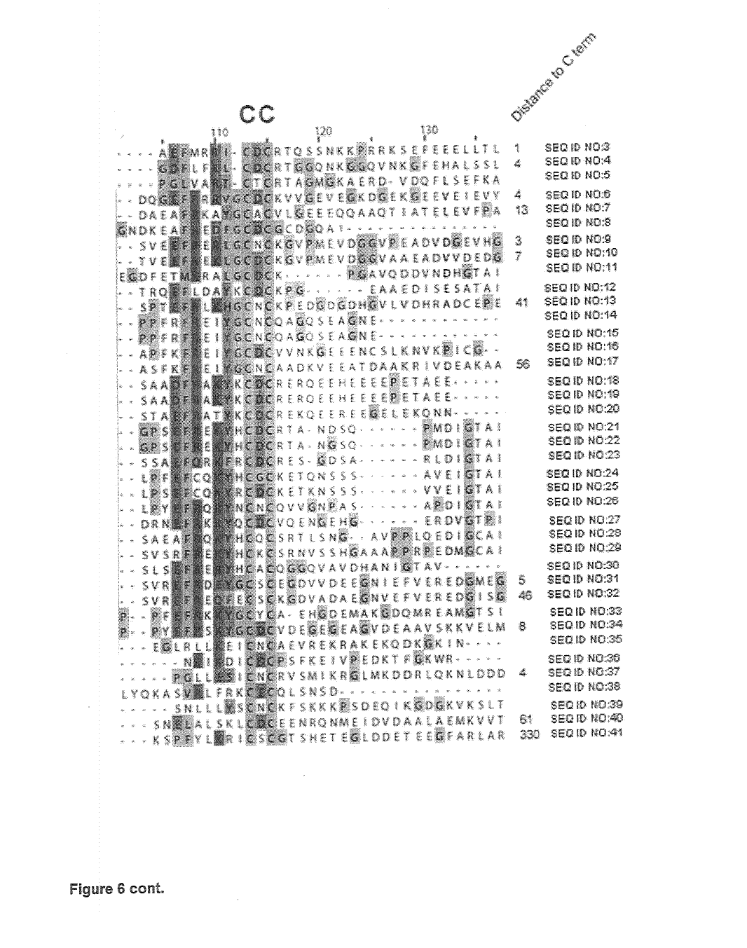

FIG. 6: Sequence alignment of the C-terminal domain of Dicer and Dicer-like protein 1
Entries in UniProtKB (release 2010_{—}10; http://www.uniprot.org/) were searched for Pfam domain PF00636 (Rnase3; http://pfam.sanger.ac.uk/). Sequences containing one or more RNase3 domain were cut after the (last) predicted RNase3 domain, and the Pfam domain PF00035 (Double-stranded RNA binding motif) was aligned to the remaining C-terminal part. The fragments were further selected for the presence of at least 15 amino acids following the putative RNA binding motif and the presence of a Cys, followed by a His followed by Cys-X-Cys distributed over the whole length of the fragment. The distances between the first Cys and the His, and the His and the Cys-X-Cys in the remaining 58 fragments were manually compared with the numbers from S. pombe and S. japonicus. Proteins originating from the same species and the same strain which were only different in 5 amino acids were reduced to one fragment as well. The multiple sequence alignment (SEQ ID NO:3-) was manually adjusted and colored (Clustalx) with Jalview (Waterhouse et al. Bioinformatics. 2009 May 1; 25(9):1189-91) and sequences to the left and the right of the RNA binding motif were removed as indicated in the figure.

### DETAILED DESCRIPTION OF THE INVENTION

The present inventors elucidated the structure of a short motif at the C-terminal of Dcr1. The present inventors noted that the action of said short motif of Dcr1 is surprisingly due to a particular structural conformation induced by a coordination complex between a cation, e.g. zinc, and 4 amino acids. The present inventors further found out that this coordination complex is present in all the fungus they investigated and that disruption of said coordination complex leads to the death of the fungal cells.

The present invention thus encompasses a method for reducing growth of a fungus having a coordination complex formed at the C-terminus of its dicer (dicer 1, ribonuclease type III) between a cation and the amino acids corresponding to the amino acids C1275, H1312, C1350 and C1352 of SEQ ID NO:1 comprising the step of contacting a fungal cell with an effective amount of an agent, wherein said agent disrupts the coordination complex formed at the C-terminus of fungal dicer between a cation and the amino acids corresponding to the amino acids C1275, H1312, C1350 and C1352 of SEQ ID NO:1. In some embodiments of the invention, the cation is selected from the group consisting of zinc, magnesium, manganese, cobalt, copper and iron. In some embodiments of the invention, the agent is an antibody or a small molecule. In some embodiments, the method of the invention is a method for treating a subject having, or being at risk of having, a fungal infection comprising the further step of administering the antifungal agent to the subject. Said subject can be a mammal, for instance a human subject or a non-human subject, or a plant or a seed.

In some embodiments of the invention, fungal cell is from the ascomycota phylum and is, for instance, selected from the group comprising Coccidioides immitis, Aspergillus niger, Aspergillus clavatus, Aspergillus oryzae, Aspergillus nidulans, Aspergillus terreus, Neosartorya fischeri, Aspergillus fumigatus, Phaeosphaeria nodorum, Magnaporthe grisea, Neurospora crassa, Chaetomium globosum, Cryphonectria parasitica, Schizosaccharomyces pombe, Cryptococcus neoformans, Coccidioides posadasii, Ajellomyces dermatitidis, Ajellomyces capsulate, Arthroderma benhamiae, Aspergillus flavus, Penicillium marneffei, Trichophyton verrucosum, Nectria haematococca, Verticillium albo-atrum, Botryotinia fuckeliana, Sclerotinia sclerotiorum, Pyrenophora tritici-repentis, Mucor circinelloides f. lusitanicus, Postia placenta, Laccaria bicolor, Coprinopsis cinerea, Schizophyfium commune H4-8, Sordaria macrospora, Podospora anserine, Nannizzia otae, Talaromyces stipitatus, Penicillium chrysogenum, Tuber melanosporum (Truffle), Schizosaccharomyces japonicas and Coprinopsis cinerea. In some embodiments, the fungal cell is from a fungus which is pathogenic for animals and is, for instance, Aspergillus fumigatus, Cryptococcus neoformans, Chaetomium globosum, Coccidioides posadasii, Coccidioides immitis, Ajellomyces dermatitidis, Ajellomyces capsulate, Arthroderma benhamiae, Aspergillus flavus, Aspergillus oryzae, Aspergillus niger, Neosartorya fischeri, Aspergillus clavatus, Aspergillus terreus, Penicillium marneffei, Trichophyton verrucosum or Nectria haematococca. In other embodiments, the fungus is a plant pathogen and is selected from a group comprising Nectria haematococca, Verticillium albo-atrum, Cryphonectria parasitica, Magnaporthe grisea, Botryotinia fuckeliana, Sclerotinia sclerotiorum, Pyrenophora tritici-repentis, Phaeosphaeria nodorum, Mucor circinelloides f. lusitanicus, and Postia placenta. A further embodiment of the invention encompasses an antibody specifically binding to an epitope at the C-terminus of a fungal dicer (dicer 1, ribonuclease type III), said epitope comprising one or more amino acid corresponding to C1275, H1312, C1350 and/or C1352 of SEQ ID NO:1, wherein the binding of said antibody to said epitope disrupts the coordination complex formed between a cation and the amino acids corresponding to C1275, H1312, C1350 and C1352 of SEQ ID NO:1 at the C-terminal of said fungal dicer.

Another embodiment of the invention encompasses an antibody, or antibody fragment, able to compete with an antibody specifically binding to an epitope at the C-terminus of a fungal dicer (dicer 1, ribonuclease type III), said epitope comprising one or more amino acid corresponding to C1275, H1312, C1350 and/or C1352 of SEQ ID NO:1, wherein the binding of said antibody to said epitope disrupts the coordination complex formed between a cation and the amino acids corresponding to C1275, H1312, C1350 and C1352 of SEQ ID NO:1 at the C-terminal of said fungal dicer.

The present invention further encompasses a method for the identification of a substance capable of disrupting the coordination complex formed between a cation and the amino acids corresponding to the amino acids C1275, H1312, C1350 and C1352 of SEQ ID NO:1 at the C-terminus of fungal dicer (dicer 1, ribonuclease type III), said method comprising the step of a) contacting said fungal dicer, or a fragment thereof wherein said fragment comprises at least the amino acids corresponding to the amino acids C1275, H1312, C1350 and C1352 of SEQ ID NO:1 and is able to form a coordination complex in presence of a cation, with a candidate substance, and b) assessing whether the coordination complex formed between the cation and the amino acids corresponding to the amino acids C1275, H1312, C1350 and C1352 of SEQ ID NO:1 is disrupted in the presence of the substance; wherein a disruption of said coordination complex indicates that said substance is a potential anti-fungal agent. In some embodiments, the integrity of said coordination complex is assessed by measuring the ability of dicer to migrate from the nucleus to the cytoplasm, for instance by use of a reporter functionally fused to said dicer. The integrity of said coordination complex can also be assessed using FRET.

The present invention also encompasses a composition comprising an antibody, or antibody fragment, according to the invention, or an agent identified using a method of the invention. Said compositions can be used as a medicament to treat a subject having, or being at risk of having, a fungal infection, or as a fungicide to treat plants having, or being at risk of having, a fungal infection.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty-1, Joining Penalty—30, Randomization Group Length=0, Cutoff Score=I, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=I, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention. Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis. Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

In one embodiment where one is assaying for the ability of a antibody to bind or compete with another one for binding to a specific epitope, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffasion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The term “epitopes,” as used herein, refers to portions of a polypeptide having antigenic or immunogenic activity in an animal, in some embodiments, a mammal, for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834, 252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In addition, in the context of the present invention, the term “antibody” shall also encompass alternative molecules having the same function, e.g. aptamers and/or CDRs grafted onto alternative peptidic or non-peptidic frames. In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CHI, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues. Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide

Antibodies may act as agonists or antagonists of the recognized polypeptides. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polycations, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716, 111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immumol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569, 825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity. Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies. Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide).

Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-I”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

By “affinity” as used herein is meant the propensity of one chemical species to separate or dissociate reversibly from another chemical species. In the present invention, the two chemical species most typically are represented by a protein and its ligand, more specifically an antibody and its target antigen. Affinity herein is measured by the equilibrium constant of dissociation (Kd or K_{d}) that defines the binding between the two chemical species. The Kd defines how tightly the species bind one another. The smaller the dissociation constant, the more tightly bound the ligand is, or the higher the affinity between ligand and protein. For example, an antigen with a nanomolar (nM) dissociation constant binds more tightly to a particular antibody than a ligand with a micromolar (μM) dissociation constant. By “greater affinity” or “improved affinity” or “enhanced affinity” or “better affinity” than a parent polypeptide, as used herein is meant that a protein variant binds to its ligand with a significantly higher equilibrium constant of association (KA or Kₐ) or lower equilibrium constant of dissociation (Kd or K_{d}) than the parent protein when the amounts of variant and parent polypeptide in the binding assay are essentially the same. For example, in the context of antibodies, a variant antibody may have greater affinity to the antigen that its parent antibody, for example when the CDRs are humanized, as described herein. Alternatively, and Fc polypeptide may have greater affinity to an Fc receptor, for example, when the Fc variant has greater affinity to one or more Fc receptors or the FcRn receptor. In general, the binding affinity is determined, for example, by binding methods well known in the art, including but not limited to Biacore™ assays. Accordingly, by “reduced affinity” as compared to a parent protein as used herein is meant that a protein variant binds its ligand with significantly lower Ka or higher Kd than the parent protein. Again, in the context of antibodies, this can be either to the target antigen, or to a receptor such as an Fc receptor. Greater or reduced affinity can also be defined relative to an absolute level of affinity. For example, greater or enhanced affinity may mean having a Kd lower than about 10 nM, for example between about 1 nM-about 10 nM, between about 0.1-about 10 nM, or less than about 0.1 nM.

The term “specifically binds” refers, with respect to an antigen to the preferential association of an antibody or other ligand, in whole or part, with a cell or tissue bearing that antigen and not to cells or tissues lacking that antigen. It is recognized that a certain degree of non-specific interaction can occur between a molecule and a non-target cell or tissue. Nevertheless, specific binding can be distinguished as mediated through specific recognition of the antigen. Although selectively reactive antibodies bind antigen, they can do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a specific antigen as compared to an unspecific antigen. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity. The present invention is also directed to antibody-based therapies which involve administering antibodies of the invention to an animal, in some embodiments, a mammal, for example a human, patient to treat a fungal infection. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-13 8 (1984)). Other controlled release systems are discussed in the review by Langer (Science 249:1527-1533 (1990)).

The present invention also provides pharmaceutical compositions for use in the treatment of fungal infection by disrupting coordination complex formed at the C-terminus of fungal dicer between a cation and the amino acids corresponding to the amino acids C1275, H1312, C1350 and C1352 of SEQ ID NO:1. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection. Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The compounds of the invention can be formulated as neutral or salt forms. Pharmaceutically acceptable salts include those formed with cations such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patients circumstances. Effective doses may be extrapolated from dose-response curves derived from in vitro or animal model test systems.

For antibodies, the dosage administered to a patient is typically 0.1 mg/kg to 100 mg/kg of the patients body weight. In some embodiments, the dosage administered to a patient is between 0.1 mg/kg and 20 mg/kg of the patient's body weight, for example 1 mg/kg to 10 mg/kg of the patients body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethy1cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

The anti-fungal agents of the invention may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The anti-fungal agents of the invention may be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The anti-fungal of the invention may also be incorporated within a slow or delayed release device. Such devices may, for example, be inserted at the site of an infection, and the molecule may be released over weeks or months. Such devices may be particularly advantageous when long term treatment is required and would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration.

Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an anti-fungal agent of the invention may be used for the treatment of an infection in the subject, depending upon which specific inhibitor is used. When the inhibitor is delivered to a cell, daily doses may be given as a single administration (e.g. a single daily injection).

Various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The present invention also provides a method of screening compounds to identify those which might be useful for fungal infection in a subject as well as the so-identified compounds.

As used herein and as in the fields of pharmacology and biochemistry, a “small molecule” is a low molecular weight organic compound which is by definition not a polymer. The term small molecule is restricted to a molecule that also binds with high affinity to a biopolymer such as protein, nucleic acid, or polysaccharide and in addition alters the activity or function of the biopolymer. The upper molecular weight limit for a small molecule is approximately 800 Daltons which allows for the possibility rapid diffuse across cell membranes so that they can reach intracellular sites of action. In addition, this molecular weight cutoff is necessary but insufficient condition for oral bioavailability. Small molecules can have a variety of biological functions, serving as cell signalling molecules, as tools in molecular biology, as drugs in medicine, and in countless other roles. These compounds can be natural (such as secondary metabolites) or artificial (such as antiviral drugs); they may have a beneficial effect against a disease (such as drugs) or may be detrimental (such as teratogens and carcinogens). Biopolymers such as nucleic acids, proteins, and polysaccharides (such as starch or cellulose) are not small molecules, although their constituent monomers—ribo- or deoxyribonucleotides, amino acids, and monosaccharides, respectively—are considered to be. Very small oligomers are also considered small molecules, such as dinucleotides, peptides such as the antioxidant glutathione, and disaccharides such as sucrose.

A fungal cell is intended to encompass any cell originating from a fungal species or fungus. As used herein a fungus is also intended to include moulds, yeast and pathogenic fungi. As used herein “reducing fungal growth” is intended to encompass an interference in fungal cell growth or processing which can be determined by a reduction in cell number, a reduction in cell division or a reduction in the yeast-to-hyphal transition phase. Methods for detecting a reduction in fungal growth are known to those of skill in the art and include high throughput assays. An example of such a method is described in PCT/US04/03208. Generally fungal cells are grown in microtitre plates and incubated with a molecule of the invention to determine reduction of fungal cell growth. For example, C. albicans cells are grown in YNB media that inhibits hyphal growth and then transferred to 384-well optical plates containing Spider media to induce the budded-to-hyphal transition and hyphal elongation. The yeast-to-hyphal morphological transition is essential for the virulence of C. albicans. An anti-fungal small molecule is added, incubated at 37° C. for 4 hours and inhibition or reduction of fungal growth determined. One of skill in the art would be able to detect a reduction in growth by routine methods such as microscopy. YNB media is well known in the art and contains yeast nitrogen base (DIFCO Labs.), glucose (US Biological) and d-H₂O, Spider media is well known in the art and contains nutrient broth (DIFCO Labs.), mannitol (Sigma-Aldrich), K₂HPO₄ (Sigma-Aldrich) and d-H₂O. Other methods for determining a reduction in fungal growth include methods for determining the number of fungal cells using cell staining techniques such as trypan blue and counting the cells using a microscope. Methods such as PCR and RT-PCR are contemplated for determining a reduction of RNA or DNA as a measure of reduced fungal growth. Other methods include observation of a visible reduction of the fungal infection as a result of reduced fungal growth. These methods are well known to those of ordinary skill in the art and require routine procedures.

As used herein subject in need thereof is a subject having a fungal infection, or a subject at risk of developing a fungal infection. The subject may have been diagnosed as having such a fungal infection as described herein or using standard medical techniques known to those of skill in the art. Alternatively a subject may exhibit one or more symptoms of fungal infection. A subject at risk of developing a fungal infection is a subject who has been exposed to a fungus, or is susceptible to exposure to a fungus. For instance a subject that is susceptible to exposure to a fungus includes those subjects who work with fungal material or in areas of high fungal content, subjects who travel to areas with high fungal infectivity rates or are otherwise likely to be exposed to a fungal infection as well as those subjects having particular susceptibility to fungal infection resulting from medical conditions or therapies. Examples of subjects having particular susceptibility to fungal infections arising from medical conditions or therapies include but are not limited to an immunocompromised subject, a subject having received chemotherapy, a subject having cancer, a subject having AIDS, a subject who is HIV positive, a subject who is at risk of being HIV positive, a subject having received a transplant, or a subject having a central venous catheter. An immunocompromised subject is a subject that is incapable of inducing a normal effective immune response or a subject that has not yet developed an immune system (e.g. preterm neonate). An immunocompromised subject, for example, is a subject undergoing or undergone chemotherapy, a subject having AIDS, a subject receiving or having received a transplant or other surgical procedure.

“Dicer”, also termed dicer 1, ribonuclease type III, DCR1, HERNA, endoribonuclease Dicer, KIAA0928, dicer 1, double-stranded RNA-specific endoribonuclease, Dicer1, Dcr-1 homolog, K12H4.8-LIKE, helicase-moi, Helicase with RNase motif, DICER, Helicase MOI, EC 3.1.26., or Dicer1, Dcr-1 homolog (Drosophila), is an endoribonuclease in the RNase III family that cleaves double-stranded RNA (dsRNA) and pre-microRNA (miRNA) into short double-stranded RNA fragments called small interfering RNA (siRNA) about 20-25 nucleotides long, usually with a two-base overhang on the 3′ end. Dicer contains two RNase III domains and one PAZ domain; the distance between these two regions of the molecule is determined by the length and angle of the connector helix and determines the length of the siRNAs it produces. Dicer catalyzes the first step in the RNA interference pathway and initiates formation of the RNA-induced silencing complex (RISC), whose catalytic component argonaute is an endonuclease capable of degrading messenger RNA (mRNA) whose sequence is complementary to that of the siRNA guide strand. The amino acid sequence of Dcr1 has the Uniprot number Q09884 and is set forth in SEQ ID NO:1. The C-terminal of Dcr1 has the amino acid sequence as set forth in SEQ ID NO:39.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting. Moreover, it is to be noted that despite that fact that the above description concentrates on medical uses, the methods and agents of the invention are also suitable for any non-medical use.

### Examples

### Strains and Plasmids:

Fission Yeast strains were grown at 30° C. in YES, as described in Emmerth et al., 2010, Dev. Cell 18, 102-113. If transformed with plasmids, strains were grown in EMMc-leu medium containing 5 μg/ml thiamine. All strains were constructed following a standard PCR-based protocol (Bahler et al., 1998, Yeast 14, 943-951). GFP-LacZ constructs were cloned by fusion PCR based assembly of the inserts and subsequent PacI/NotI ligation into pREPN-3×FLAG (Buker et al., 2007, NatStructMol Biol 14, 200-207). pREP1-3×FLAG-Dcr1ΔC33 was constructed by exchanging Dcr1 with Dcr1ΔC33 by BgIII/NotI cloning into pREP1-3×FLAG-Dcr1 (Colmenares et al., 2007, Mol Cell 27, 449-461). Constructs on plasmids and in yeast strains were confirmed by sequencing.

### Yeast Live Cell Fluorescence Microscopy:

If not specified differently, S. pombe precultures were grown in YES (sterile filtered components only) for 8 hours at 30° C., diluted to 10⁵ cells/mL and grown for 14-16 hours at 30° C. to a concentration of about 5·10⁶ cells/mL. Microscopy was performed on cells spread on agarose patches containing YES medium with 3% glucose. Images were captured on a Delta Vision built of an Olympus IX70 widefield microscope equipped with a CoolSNAP HQ2/ICX285 camera. Image stacks of 12-15 μm Z-distance were acquired with a Z-step size of 0.2 μm and deconvolved using the softworks (Delta Vision) software.

### Confocal Microscopy and FLIP:

Cells expressing the plasmid-encoded fusion proteins GFP-lacZ, GFP-lacZ+dsRBD and GFP-lacZ+dsRBD+C33 where grown in PMGc-Leu supplemented with 1 μg/mL thiamine for 6-8 h. From this preculture cells where diluted into fresh medium to a concentration of 7×10⁵ cells/mL and grown for 14 h. Strains with genomic modifications of the dcr1+ locus were grown in YES for 6-8 h. This preculture was diluted to 0.5−1×10⁵ cells/mL into fresh medium and grown for 14 h. G2 Cells were imaged in liquid media using a ludin chamber and Bandeiraea simplicifolia (Sigma L7508, 1 mg/mL solution) coated cover slips. Bleaching was performed on a Zeiss LSM510 microscope. The pinhole was set to 1 airy unit corresponding to 0.7 μm optical thickness. The mean fluorescence of nuclei was determined using the ImageJ software. The values where normalized to control neighbouring nuclei and to the first time point.

RNA Isolation and cDNA Synthesis for qRT-PCR:

Cells were harvested at OD₆₀₀=0.5, washed once in water and flash frozen in liquid nitrogen. RNA was isolated using the Absolutely RNA® Miniprep Kit from Stratagene (#400805) with the following modification: 600 μl lysis buffer, 4.2 μl Mercaptoethanol and 500 μl glass beads were added to the pellet and cells were bead-beaten for 1 min. The cleared lysate was collected and RNA isolation was continued according to the supplier's manual. cDNA was synthesized using random hexamers with the AffinityScript MultipleTemperature cDNA Synthesis Kit from Stratagene (#200436).

### Quantitative RT-PCR/PCR:

Quantitative PCR was performed on a 7500 Fast Real-Time PCR System (Applied Biosystems #4351106) using the Fast SYBR® Green Master Mix (Applied Biosystems #4385612). Relative RNA levels were calculated from C_{T} values according to the ΔC_{T} method and normalized to act1+ mRNA levels. Primer pairs used for PCR reactions can be found in supplemental experimental procedures.

### Silencing Assays:

Serial 10-fold dilutions of the strains indicated were plated on PMGc (non selective, NS) or on PMGc plates containing 2 mg/mL 5-FOA.

### Chromatin Immunoprecipitation (ChIP) and Northern Blot:

ChIP was performed as described in (Buhler et al., 2006, Cell 125, 873-886). An antibody against dimethylated H3-K9 (abcam, #ab1220) was used. Centromeric siRNA were detected by Northern blotting as described in (Buhler et al., 2006, Cell 125, 873-886).

### The C-Terminus of Dcr1 Mediates Nuclear Localization and is Required for Heterochromatin Assembly:

Fission yeast Dcr1 contains an N-terminal helicase/ATPase domain, followed by a DUF283, two RNAseIII domains and a long C-terminal domain that bears a divergent dsRBD (Colmenares et al., 2007, Mol Cell 27, 449-461). Importantly, the helicase, RNAseIII, and C-terminal domains are all critical for centromeric silencing and RNAi in vivo, whereas the C-terminus is not required for dsRNA processing in vitro (Colmenares et al., 2007, Mol Cell 27, 449-461). One part of Dcr1 that is required for silencing in vivo but is not essential for dsRNA processing in vitro is its 103 amino acid long C-terminus (C103; Colmenares et al., 2007, Mol Cell 27, 449-461). This C-terminus contains the enzyme's dsRBD and a 33-amino acid extension (C33). To test the possibility that C103 mediates subcellular localization of Dcr1, some of the inventors generated in a previous study (Emmerth et al., 2010, Dev. Cell, 18, 102-113) a strain expressing GFP-tagged Dcr1 lacking C103 (GFP-Dcr1ΔC103). In contrast to full length GFP-Dcr1, GFP-Dcr1ΔC103 localization was mainly cytoplasmic. To determine the contribution of C33, they repeated this analysis on GFP-tagged Dcr1 lacking C33. Importantly, deletion of C33 resulted in the same if not even stronger loss of nuclear localization phenotype as observed for the C103 deletion without affecting protein stability (GFP-Dcr1ΔC33). Loss of heterochromatic gene silencing has been previously demonstrated for Dcr1ΔC103 (Colmenares et al., 2007, Mol Cell 27, 449-461). Similarly, silencing of centromeric heterochromatin and the generation of centromeric siRNAs was abolished in cells expressing Dcr1ΔC33. The observed loss of silencing was due to a failure in assembly of heterochromatin at centromeric repeats because H3K9 methylation was also affected in dcr1ΔC33 cells. Thus, the short C-terminal motif (C33) mediates nuclear localization of Dcr1 and is required for the assembly of heterochromatin at centromeric repeats.

### Dcr1ΔC33 is a Shuttling Protein:

Relocation of C-terminally truncated Dcr1 (GFP-Dcr1ΔC33) from the nucleus to the cytoplasm led to the hypothesis that C33 could act as a nuclear localization signal (NLS). To directly test this, some of the inventors fused in a previous study (Emmerth et al., 2010, Dev. Cell, 18, 102-113) C33 to a GFP-LacZ reporter construct, which had previously been shown to localize throughout the cell (Dang and Levin, 2000, Mol Cell Biol 20, 7798-7812). In contrast to the SV40 NLS, S. pombe C33 did not lead to an enhanced nuclear localization of the GFP-LacZ reporter. Therefore, C33 is not sufficient for nuclear localization, and, hence, is unlikely to be a NLS. Surprisingly, although the SV40 NLS was sufficient for nuclear accumulation of a GFP-LacZ reporter, this sequence added either C- or N-terminally, was not able to restore nuclear localization of GFP-Dcr1ΔC33. Moreover, the addition of a C-terminal NLS to Dcr1ΔC33 was unable to restore silencing when expressed from Dcr1's endogenous promoter. However, strong overexpression of Dcr1ΔC33 fully rescued loss of silencing of a centromeric ura4⁺ reporter. Furthermore, partial rescue of heterochromatic gene silencing upon mild overexpression of Dcr1ΔC33 was observed, which was further improved by adding an SV40 NLS. These results strongly suggested that the SV40 NLS did promote nuclear import of Dcr1ΔC33, although it never accumulated to high levels in the nucleus due to faster export kinetics. To directly test this hypothesis, fluorescence loss in photobleaching (FLIP) experiments were performed, where the cytoplasm of cells expressing GFP-Dcr1ΔC33 or NLS-GFP-Dcr1ΔC33 was bleached while measuring fluorescence in the nucleus. Cytoplasmic bleaching resulted in a rapid decrease of nuclear fluorescence, demonstrating that GFP-Dcr1ΔC33 and NLS-GFP-Dcr1ΔC33 are shuttling proteins.

In conclusion, the results of said previous study (Emmerth et al., 2010, Dev. Cell, 18, 102-113) demonstrate that fission yeast Dcr1 has the ability to shuttle between the nucleus and the cytoplasm and is very efficiently exported to the cytoplasm if the 33 most C-terminal amino acids are missing. Therefore, rather than acting as a NLS, C33 functions as a nuclear retention signal by inhibiting nucleo-cytoplasmic export of Dcr1.

The present inventors performed further studies to determine the exact mechanism of action of C33. During these further studies, the present inventors solved the structure of the Dcr1 C-terminus and surprisingly noted that 4 specific amino acids of C33 form a coordination complex with a Zinc molecule. The formation of this coordination complex at the C terminus of Dcr1 is crucial for the formation of heterochromatin and growth of fission yeast, as the mutation of any one of the amino acids involved in the coordination complex induces the shuttling of Dcr1 similar to the one observed in the absence of the C33.

Moreover, the present inventors also realized that, as compared to e.g. human Dicer, fungal Dcr1 comprise a C-terminal elongation similar to the C33 of fission yeast, and that the amino acids crucial for the formation of the coordination complex are conserved in said fungal Dcr1.

In view of their further finding that fission yeast having a mutation in any one of the amino acids involved in the coordination complex cannot grow, the inventors realized that this coordination complex is a prime target for the development of an anti-fungal agent.

1. A method for reducing growth of a fungus having a coordination complex formed at the C-terminus of its dicer (dicer 1, ribonuclease type III) between an anion and the amino acids corresponding to the amino acids C1275, H1312, C1350 and C1352 of SEQ ID NO:1 comprising the step of contacting a fungal cell with an effective amount of an agent, wherein said agent disrupts the coordination complex formed at the C-terminus of fungal dicer between an anion and the amino acids corresponding to the amino acids C1275, H1312, C1350 and C1352 of SEQ ID NO:1.

2. The method of claim 1 wherein the anion is selected from the group consisting of zinc, magnesium, manganese, cobalt, copper and iron.

3. The method of claim 1 wherein the agent is an antibody or a small molecule.

4. The method of claim 1 wherein the method is a method for treating a subject having, or being at risk of having, a fungal infection comprising the further step of administering the antifungal agent to the subject.

5. The method of claim 4 wherein the subject is a mammal.

6. The method of claim 4 wherein the subject is a plant or a seed.

7. The method of claim 1 wherein the fungal cell is from the ascomycota phylum and is selected from the group consisting of Coccidioides immitis, Aspergillus niger, Aspergillus clavatus, Aspergillus oryzae, Aspergillus nidulans, Aspergillus terreus, Neosartorya fischeri, Aspergillus fumigatus, Phaeosphaeria nodorum, Magnaporthe grisea, Neurospora crassa, Chaetomium globosum, Cryphonectria parasitica, Schizosaccharomyces pombe, Cryptococcus neoformans, Coccidioides posadasii, Ajellomyces dermatitidis, Ajellomyces capsulate, Arthroderma benhamiae, Aspergillus flavus, Penicillium marneffei, Trichophyton verrucosum, Nectria haematococca, Verticillium albo-atrum, Botryotinia fuckeliana, Sclerotinia sclerotiorum, Pyrenophora tritici-repentis, Mucor circinelloides f. lusitanicus, Postia placenta, Laccaria bicolor, Coprinopsis cinerea, Schizophyllum commune H4-8, Sordaria macrospora, Podospora anserine, Nannizzia otae, Talaromyces stipitatus, Penicillium chrysogenum, Tuber melanosporum (Truffle), Schizosaccharomyces japonicas and Coprinopsis cinerea.

8. The method of claim 1 wherein the fungal cell is a fungus for animals.

9. An antibody specifically binding to an epitope at the C-terminus of a fungal dicer (dicer 1, ribonuclease type III), said epitope comprising one or more amino acid corresponding to C1275, H1312, C1350 and/or C1352 of SEQ ID NO:1, wherein the binding of said antibody to said epitope disrupts the coordination complex formed between an anion and the amino acids corresponding to C1275, H1312, C1350 and C1352 of SEQ ID NO:1 at the C-terminal of said fungal dicer.

10. An antibody, or antibody fragment, specifically binding to fungal dicer (dicer 1, ribonuclease type III) characterized in that said antibody competes with an antibody of claim 9 for the binding to fungal dicer.

11. A method for the identification of a substance capable of disrupting the coordination complex formed between an anion and the amino acids corresponding to the amino acids C1275, H1312, C1350 and C1352 of SEQ ID NO:1 at the C-terminus of fungal dicer (dicer 1, ribonuclease type III), said method comprising the step of:
a) contacting said fungal dicer, or a fragment thereof wherein said fragment comprises at least the amino acids corresponding to the amino acids C1275, H1312, C1350 and C1352 of SEQ ID NO:1 and is able to form a coordination complex in presence of an anion, with a candidate substance, and
b) assessing whether the coordination complex formed between the anion and the amino acids corresponding to the amino acids C1275, H1312, C1350 and C1352 of SEQ ID NO:1 is disrupted in the presence of the substance, wherein a disruption of said coordination complex indicates that said substance is a potential anti-fungal agent.

12. The method of claim 11 wherein the integrity of said coordination complex is assessed by measuring the ability of dicer to migrate from the nucleus to the cytoplasm.

13. The method of claim 11 wherein the integrity of said coordination complex is assessed using FRET.

14. A composition comprising an antibody, or antibody fragment, according to claim 9.

15. (canceled)

16. The method of claim 5 wherein the mammal is a human subject.

17. The method of claim 8, wherein the fungus is selected from the group consisting of Aspergillus fumigatus, Cryptococcus neoformans, Chaetomium globosum, Coccidioides posadasii, Coccidioides immitis, Ajellomyces dermatitidis, Ajellomyces capsulate, Arthroderma benhamiae, Aspergillus flavus, Aspergillus oryzae, Aspergillus niger, Neosartorya fischeri, Aspergillus clavatus, Aspergillus terreus, Penicillium marneffei, Trichophyton verrucosum, Nectria haematococca, Nectria haematococca, Verticillium albo-atrum, Cryphonectria parasitica, Magnaporthe grisea, Botryotinia fuckeliana, Sclerotinia sclerotiorum, Pyrenophora tritici-repentis, Phaeosphaeria nodorum, Mucor circinelloides f. lusitanicus, and Postia placenta.

18. The method of claim 12, wherein the coordination complex is assessed by use of a reporter functionally fused to said dicer.

19. An agent identified using the method of claim 11.
